SEQUENCE LISTING (9 sequences)

SEQ ID NO: 1 - DNA, 21 bp, Artificial
  Forward Primer for Real-Time PCR
agcctggatctccttggtgac

SEQ ID NO: 2 - DNA, 18 bp, Artificial
  Reverse Primer for Real-Time PCR
accgccagtctcctgcct

SEQ ID NO: 3 - DNA, 19 bp, Artificial
  LNA-modified MGB Probe, fluorochrome labelled, specific for non-mutated sequence (Wild-Type)
ctgctgaggtggcagtttc

SEQ ID NO: 4 - DNA, 19 bp, Artificial
  LNA-modified MGB Probe, fluorochrome labelled, specific for mutated sequence 515W>L
ctgctgaggttgcagtttc

SEQ ID NO: 5 - DNA, 22 bp, Artificial
  LNA-modified MGB Probe, fluorochrome labelled, specific for mutated seauence 515W>K
tgctgctgaggaagcagtttcc

SEQ ID NO: 6 - DNA, 20 bp, Artificial
  Forward Primer for direct sequencing reaction
tagcctggatctccttggtg

SEQ ID NO: 7 - DNA, 20 bp, Artificial
  Reverse Primer for direct sequencing reaction
agaggtgacgtgcaggaagt

SEQ ID NO: 8 - DNA, 21 bp, Artificial
  Forward Primer for cloning the MPL gene fragment of interest
tgggccgaagtctgacccttt

SEQ ID NO: 9 - DNA, 28 bp, Artificial
  Reverse Primer for cloning the MPL gene fragment of interest
agaggtgacgtgcaggaagtggcgaagc

(19) United States
(12) Patent Application Publication
(10) Pub. No.: US 2011/0020805 A1
(43) Pub. Date: Jan. 27, 2011

(54) MUTATIONAL ANALYSIS OF CHRONIC MYELOPROLIFERATIVE DISORDERS

(76) Inventors: Alessandro Maria Vannucchi, Sesto Fiorentino (Firenze) (IT); Alessandro Pancrazzi, Figline Valdarno (Firenze) (IT); Paola Guglielmelli, Pistoia (IT); Vanessa Ponziani, Pieve A Nievole (Pistoia) (IT)

Correspondence Address: Steinfl & Bruno, 301 N Lake Ave Ste 810, Pasadena, CA, 91101, US

(21) Appl. No.: 12/746,515

(22) PCT Filed: Dec. 5, 2008
(86) PCT No.: PCT/IB08/55122

§ 371 Date: Oct. 4, 2010

(30) Foreign Application Priority Data
Dec. 7, 2007 (IT) FI2007A000274

(51) Int. Cl. C12Q 1/68; C12N 15/63; C07H 21/04

(52) U.S. Cl. 435/6; 435/300.1; 536/23.1; 536/24.3; 536/24.33

(57) ABSTRACT

The invention relates to molecular assays, reagents and kit for the mutational analysis, for diagnostic and prognostic purposes, of chronic myeloproliferative disorders, a group of neoplastic pathologies of the haemopoietic system. The invention relates to the identification of nucleic acid probes labelled with fluorochrome, allowing a quantitative assessment, in a specific and sensitive way, of mutation of MPL gene sequence and quantification of the mutated alleles of the MPL gene in Genomic DNA samples from patients with chronic myeloproliferative syndrome.

### TECHNICAL FIELD OF THE INVENTION

The invention regards molecular assays, reagents and kits, for mutational analysis—for diagnostic and prognostic purposes—of chronic myeloproliferative disorders, a group of neoplastic pathologies of the haemopoietic system. The invention regards the identification of nucleic acid probes, modified according to the LNA (locked nucleic acid) technology and labelled with fluorochrome, which allow the quantitative determination—in a specific and sensitive manner—, of mutations of the MPL (Myelo Proliferative Leukemia) gene sequence and quantification of the mutated alleles of the MPL gene in samples of Genomic DNA obtained from patients with chronic myeloproliferative syndrome.

### BACKGROUND ART

Chronic myeloproliferative disorders (MPD) are a heterogeneous group of hematologic diseases caused by the neoplastic transformation of a pluripotent stem cell, and comprise various nosologic entities the most common being Polycythemia Vera (PCV), Essential Thrombocythemia (ET) and Primary Myelofibrosis (PMF). These are some of the most common onco-hematological pathologies.

Up to mid 2005, MPD diagnosis was exclusively based on clinical and histopathological (bone marrow biopsy) criteria, with considerable difficulties especially concerning distinction between various clinical forms indicated above; the situation changed then, when four different research groups simultaneously described the presence of a point mutation of the JAK2 gene (JAK2 617V>F).

Subsequently, using screening genomic techniques, a second recurrent mutation, concerning the MPL gene was identified (Pikman Y, Lee B H, Mercher T, et al. MPLW515L is a novel somatic activating mutation in myelofibrosis with myeloid metaplasia. PLoS Med. 2006; 3:e270).

This gene codes for the receptor, called MPL, of Thrombopoietin (THPO); the latter is a hematopoietic growth factor which for a long period of time has been suspected, according to experimental results and animal models, of playing a role in the pathogenesis of these diseases and in particular in the PMF. The MPL receptor is a transmembrane protein belonging to the large family of the hematopoietic cytokine receptors. The coding gene is located on chromosome 1p34 and it is made up of 12 exons.

In the first study that revealed this genetic disorder, the presence of a point mutation at the nucleotide 30267 level of the MPL gene (ref seq. AL139289) corresponding to position 1544 of the mRNA sequence (ref. seq. Hs 82906, Pikman et al,), amino acid 515 of the protein (codon 515), which determined the substitution of a guanine with a thymine was demonstrated, in patients with PMF, such patients having resulted negative to the JAK2V617F mutation research. This identification was performed using the direct sequencing technique. The mutation is reflected at proteic level in the substitution of a tryptophan with a leucine in position 515 (MPLW515L). Comparative analysis of the DNA taken from cells of the mouth mucosa demonstrated that MPLW515L is a somatic mutation present solely in the hematopoietic cells derived from the neoplastic clone (Pikman Y, et al supra).

In order to evaluate the effects of the mutation, and thus be able to clarify its role in the pathogenesis of the disease, murine models expressing MPLwild-type (MPLWT) and MPLW515L were created through transfection experiments of the haemopoietic cells transplanted into the recipient. In mice expressing the mutated receptor, the development of a lethal myeloproliferative disease was observed, characterised by a marked increase of platelets and white cells, enlargement of the spleen volume (splenomegaly) and liver (hepatomegaly) and the development of fibrosis at the bone marrow tissue level, thus reproducing the characteristic phenotype of the human disease (Pikman Y, et al. supra). Furthermore, it was demonstrated that the MPLW515L mutation induces autonomous growth capacity—which is a fundamental characteristic of tumour cells—in the cell.

In a subsequent study, a further mutation—called MPL 515W>K—was identified, still at codon 515 level. This is a double nucleotide substitution which modifies the codon from TGG of the normal gene to AAG in the mutated gene, determining a substitution from tryptophan to lysine in the amino acid chain (Pardanani A D, Levine R L, Lasho T, et al. MPL515 mutations in myeloproliferative and other myeloid disorders: a study of 1182 patients. Blood. 2006; 108:3472-3476). Even this mutation causes constitutive activation of the MPL receptor, and thus of the signalling path downstream of the same, which leads to the same alteration of the growth property of the hematopoietic cells configuring the tumour phenotype. In this second study, the research of mutations of codon 515 of the MPL gene was partially performed using the direct sequencing method, like in the previous case, and partially using an alternative method based on the melting curves. The sensitivity level of this second approach, according to the information provided in the publication, was 3%-5% of mutated allele mixed with a healthy allele.

Generally, according to these two studies, the MPLW515UK mutations were present at a frequency of 5% in patients with PMF and 1% in patients with ET, while as for now there are no cases detected in patients affected by PCV (Pardanani A D, et al. supra). More recent studies have shown that the JAK2V617F and MPLW515UK mutations may coexist in patients affected by PMF (Lasho T L, Pardanani A, McClure R F, et al. Br. J. Haematol. 2006; 135:683-687). Using direct sequencing, the allele load (i.e. the % of mutated allele with respect to the total) of the two mutations present in the same subject was evaluated in a semi-quantitative manner, and it was observed that a larger MPL mutant clone is usually accompanied by a less relevant JAK2 mutant clone. Both mutations seem to be early events of the disease and it is hypothesised that the phenotype variations are due not only to the presence of the mutation, but above all to the fluctuations of the allele load over time. The latter observation emphasizes on the importance of the possibility of having a quantitative method capable of allowing a sensitive analysis of the mutations of the allele load of the mutated gene, which is one of the advantages obtainable from the use of this invention with respect to the conventional method of direct sequencing.

Lastly, Guglielmelli et al. published a study on over 200 patients with PMF, in which it was demonstrated, using the direct sequencing method, that the MPL mutations are associated to a more aggressive clinical phenotype linked to a greater transfusion dependence with respect to patients not showing any mutation or mutated in JAK2. (Guglielmelli P, Pancrazzi A, Bergamaschi G, et al. Br. J. Haematol. 2007; 137:244-247). Therefore, the results of this study further support the importance of diagnosis aimed at the mutations of codon 515 of the MPL gene in that, in the category of patients with PMF, they can represent a negative prognostic factor, potentially influencing the therapeutic choices for the management of the patients themselves.

According to the information provided above it is clear that the techniques used up to now for the research of mutations of codon 515 of the MPL gene in patients with chronic myeloproliferative diseases were based on direct sequencing (Pikman Y, Lee B H, Mercher T, et al. PLoS Med. 2006; 3:e270) or on the use of melting curves (Pardanani A D, Levine R L, Lasho T, et al. Blood. 2006; 108:3472-3476).

It is believed that direct sequencing methods have low sensitivity, in that they are not capable of detecting the mutated allele if present by less than 15% approximately in a mixture of normal and mutated alleles. This low sensitivity represents a problem in particular in diseases such as chronic myeloproliferative diseases, given that cells of the neoplastic clone, which carry the mutation of the MPL gene, may not represent the totality of the blood cells, but only a percentage, sometimes even a very small percentage.

The second methodological approach has higher sensitivity with respect to direct sequencing, and it amounts to approximately 5%, depending on the characteristics of the probes and on the efficiency of the amplification reaction. The methodology requires a commonly available apparatus, but its interpretation is not easy and above all, the quantification of the ratios between the mutated sequence and the wild-type sequence is not easily standardizable or reproducible.

In conclusion, research of 515W>L and 515W>K mutations of the MPL gene has been performed up to date through techniques hindered by procedural and interpretation difficulties, as well as low sensitivity.

Therefore, the object of the present invention is to provide new instruments and methods for mutational analysis for efficient identification of the presence of the two mutations of the MPL gene in Genomic DNA samples and for quantifying the percentage amount of mutated allele (allele load) and the possible fluctuations of such amount over time with respect to the unmutated gene, both spontaneously during the progress of the disease and in response to the treatments employed.

### SUMMARY OF THE INVENTION

The present invention is based on the observation, made by the inventors, that the techniques employed up to date allow neither an accurate detection of the presence of mutations on codon 515 of the MPL gene nor the quantitative determination of the amount of mutated allele. Furthermore, experiments performed by the same inventors showed that not even the standard technique of amplification with Real Time Quantitative PCR and conventional probes offered satisfactory results in quantifying the allele load of the mutated gene.

The solution proposed by the invention consists in an analytical methodology based on the Real-Time Quantitative PCR (RTQ-PCR) technology integrated in a system of molecular reagents suitable for such purpose. In particular, the essential and innovative characteristic of the invention is represented by the identification and optimisation of molecular probes, developed through the LNA (Locked Nucleic Acids) technology, said probes being capable of discriminating between the three different alleles of the MPL gene. The probes turned out to be the key element for the development of a sensitive and specific diagnostic or prognostic analytical system.

Therefore, a first object of the present application are labelled genetic probes for determining the presence of mutations on codon 515 of the MPL gene and the amount of mutated allele, containing at least one nucleotide modified through the LNA (locked Nucleic Acid) technique. In particular, probes in which codon 515 of the native MPL gene (wt) is codon TGG coding for the tryptophan amino acid and in which codon 515 of the mutated allele is a codon respectively coding for a leucine or lysine residue.

A second object are the primers for amplifying the fragments of the MPL gene comprising codon 515.

A third object is an in vitro mutational analysis method for determining the presence of mutations on codon 515 of the MPL gene and the amount of mutated allele, wherein a sample of Genomic DNA containing the gene is subjected to amplification through the RTQ-PCR (Real Time Quantitative PCR) technique in the presence of one or more probes according to the invention.

A fourth object is the application of such method for molecular diagnosis and/or for prognostic evaluation of chronic myeloproliferative diseases, as well as a reagents Kit for implementing the method. Further objects shall be clear in light of the following detailed description.

### ADVANTAGES

The invention allows overcoming the limitations of the current method, in that: 1) the technology according to the invention is within reach for most of the diagnostic laboratories; 2) it is extremely sensitive, allowing the detection of ≦0.1% of mutated allele; 3) due to its performance characteristics, it is suitable for studies on wide ranges of case histories of the patients; 4) it has lower costs; 5) it does not require specific apparatus, analytical software, neither specific know-how except for the basic ones required for setting up common analytical reactions for amplifying nucleic acids.

### DESCRIPTION OF THE FIGURES

FIG. 1: the figure shows the fluorescence trend, associated to the increase of the specific amplification product, in a typical RTQ-PCR reaction.

FIG. 2: the figure shows the performance of the amplification system with RTQ-PCR singularly referring to each plasmid, corresponding to the wild-type, mutated 5151W>L and 5151W>K sequence. The upper part of the figure shows the “amplification plot” curves obtained using progressive dilutions of the respective plasmids comprised in the range of 1-106 copies. The lower part of the figure shows the regression curves, with the parameters indicated for each one of them, obtained by correlating the number of plasmids with the CT value. Results of 5 different experiments; the standard deviation is not indicated in that it is comprised within the thickness of the symbols.

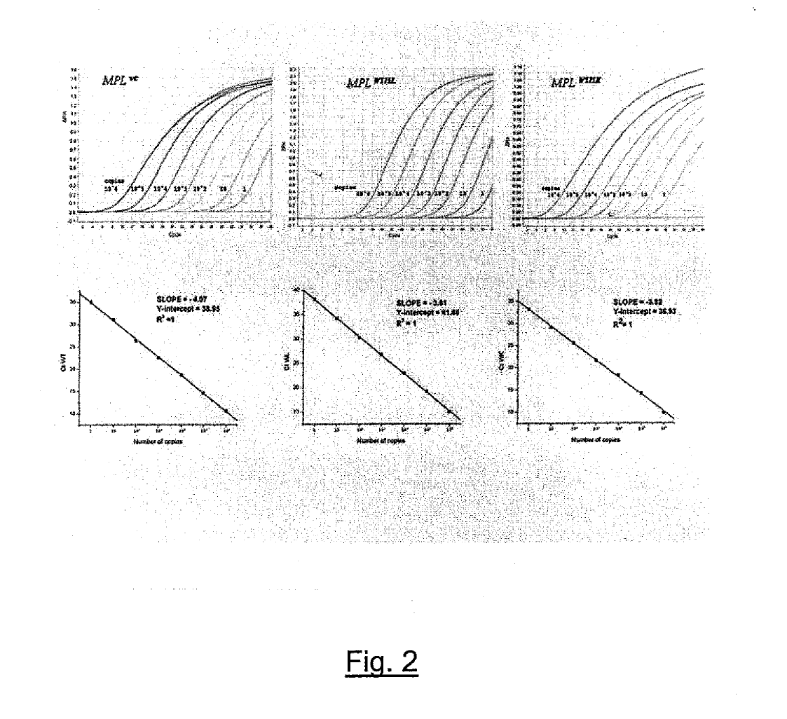

FIGS. 3A and B: The figure describes the calibration curves for the mutation 515W>L (FIG. 2A) and 5151W>K (FIG. 2B), as they are used in the analytical experimental procedure. Serial dilutions of mutated plasmid were prepared in a 1% to 100% ratio in a mixture of 99% to 0% plasmid of the wild-type allele. The entire figure shows the calibration curve from 0% to 100% of mutated allele W>L and W>K, while the smaller panels describe the regression line in the range of 0-10% of mutated allele. Results of 10 different experiments; the standard deviation is not indicated in that it is comprised within the thickness of the symbols.

FIG. 4: the figure indicates the exact position of the primers and of the amplified zone on the total sequence of the MPL gene.

### DETAILED DESCRIPTION OF THE INVENTION

### Diseases

The invention is a new analytical technology for diagnostic and possibly prognostic purposes, applicable to the study of some hematological neoplasms being part of the chronic myeloproliferative diseases (MPD), in particular Primary Myelofibrosis (PMF), Polycythemia Vera (PCV) and Essential Thrombocythemia (ET).

Cells

Specifically, this method is based on the quantitative amplification of Genomic DNA, extracted from hematopoietic cells, typically peripheral blood granulocytes although other types of cells such as bone marrow aspirations can be used.

Gene

In order to identify the sequence of the pairs of primers and of probes subject of this invention, the nucleotide sequence published in Entrez Nucleotide corresponding to LOCUS AL139289.6 was used. This is a human DNA sequence of 90433 bp, clone RP1-92014 on chromosome 1p33-34.2, which contains the complete sequence of MPL (MyeloProliferative Leukemia Virus Oncogene). The choice of the primers and of the probes sequence was performed using the type of software commonly available to the public such as Primer3 or primer Express (Applied Biosystems), and further verified with the MELT-CALC, Ver 2.0 software.

Mutation

The method of the invention represents a typical analysis of the SNP type, in that it allows discriminating between the normal sequence of codon 515 of the MPL gene (TGG, coding for the tryptophan amino acid) and the mutated sequences respectively of type L (TTG, coding for the leucine amino acid) or K (AAG, coding for the lysine amino acid); as a matter of fact, at the level of codon 515 of MPL there occurs the substitution of only 1 base in case of mutation of the W>L type and two bases in case of mutation of the W>K type (the mutated base/s are underlined).

RTQ PCR

The RTQ-PCR is a method for amplifying nucleic acids which allows their quantification in an extremely sensitive and reproducible manner. it is based on the principle of “polymerase chain reaction (PCR)”, a nucleic acid amplification technology. It represents the most advanced molecular diagnostic method and it is the most commonly used, and characterised by high specificity and sensitivity.

The PCR (just like the RTQ-PCR) is an amplification technique for subsequent cycles of nucleotide fragments. It is based on the action of the polymerase DNA, which—once activated—catalyses the synthesis and thus the amplification of the selected fragment starting from a pair of primers which delimit the same fragment.

The amplification carried out in a reaction mixture containing a buffered solution with an appropriate concentration of salts, in particular magnesium chloride; the deoxyribonucleotide triphosphates (cytosine, guanine, adenine and thymine) and a thermostable polymerase DNA enzyme. The segment of amplified DNA , comprised between the two primers, is referred to as “amplicon”.

In the conventional PCR, the detection of the amplicon generated in the amplification reaction occurs through electrophoretic separation in agarose gel, or polyacrylamid. However, this separation technique does not provide quantitative results (it can only be said that the amplicon was formed, that it is visually “a lot” or “little”, but a quantitative estimation cannot be provided).

The “Real Time Quantitative PCR” (RTQ-PCR) is a variant of the conventional PCR technique differing from the latter in that alongside the pair of specific primers, one (or even more) nucleotide sequences serving as a probe, are added to the reaction mixture. In practice, the probes are oligonucleotides, of 10-20 nucleotides, which are complementary to a segment of the DNA sequence which is comprised between the two primers, hence in the amplicon; this probe has chemical modifications which allow the quantification of the amplified product. Mainly, there are two types of quantitative RT PCR: the “Real Time” system (Applied Biosystems, AB), described as preferred in this application and the “Light Cycler” (Roche). Both are based on the direct determination of the DNA which was fluorescently-labelled with a non-specific tracer for the amplicon (SYBRgreen) or with a fluorescent primer or with a fluorescent probe.

The “Real time” mechanism is not different from a normal thermocycler, in that it provides for that the amplification reaction occurs in test tubes or plates, being provided with a laser ray which—in the various wells with the test tubes—reads the fluorescence increase obtained during the amplification reaction (Real-Time determination). On the other hand, the “Light Cycler” system, provides for that the PCR reaction occurs in a capillary inserted in a rotor which rotates with respect to the laser reader, which is stationary, contrary to the Applied Biosystem system wherein the laser ray is moved to record the fluorescence level emitted in the various test tubes. Both systems operate through the FRET (fluorescence resonance energy transfer) method based on the use of a probe with a quencher molecule in 5′ and a fluorophore present on a reverse primer (position 3′).

In the preferred embodiment of the invention, the oligonucleotide serving as a probe contains—at the 5′ end—the reporter which is a fluorescent marker (for example, FAM dye) and at the 3′ end a quencher (TAMRA dye), covalently bound. The reaction exploits the 5′ exonucleasic activity of the Polymerase DNA which allows, during the amplification, cutting the probe; in turn, this determines the removal of the quencher from the reporter and the emission of fluoroscence by the latter. Thus, there will be an accumulation of fluorescence—at each cycle of the reaction—corresponding to the accumulation of the PCR products. The increase of the fluorescence emitted by the reporter is recorded by a specific instrument, such as ABI Prism 7300 Sequence Detection System or OneStep Taqman system, both of Applied Biosystem, which were used during our study. The principle on which the phenomenon of progressive accumulation of the fluoroscent signal in the system is based is the following: when the probe is integral, the fluorescence of the reporter is absorbed by the quencher, due to the Förster-type energy transfer phenomenon (also referred to as Fluorescence Resonance Energy Transfer, FRET). During the amplification reaction, assuming that the DNA sequence intended to be amplified is present, it shall occur that the labelled probe shall hybridize in a specific manner according to the complementary sequence of the amplicon at each amplification cycle. Subsequently, the 5′-3′ exonucleasic activity of the enzyme cuts the probe separating the reporter from the quencher and thus determining the fluoroscence emission. This exclusively occurs if the probe is perfectly hybridized to the complementary sequence of the amplicon, which makes the reaction very specific. Once the probe is cut, its fragments are detached from the target and the amplification proceeds beyond up to the completion of the amplicon segment comprised between the two primers. The buffer used for the PCR reaction in the described conditions also contains a passive reference marker (ROX) which does not take part in the PCR reaction. It serves as an internal reference standard by means of which the signal of the reporter can be normalized during the data analysis. Normalisation is required to correct fluorescence fluctuations due to changes in concentrations or volumes of the samples. Normalisation is obtained from dividing fluorescence emission intensity of the reporter by the passive marker emission intensity, obtaining a ratio defined as Rn (normalised reporter) for each test tube in which the PCR reaction occurs.

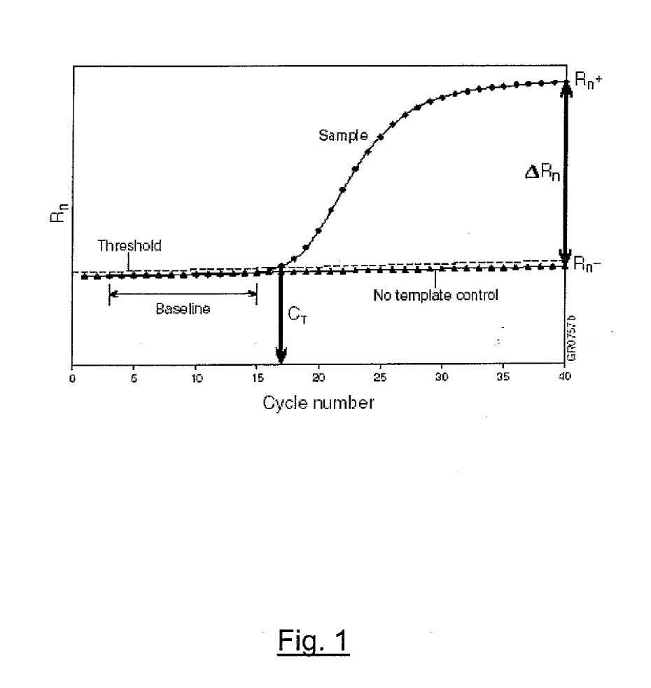

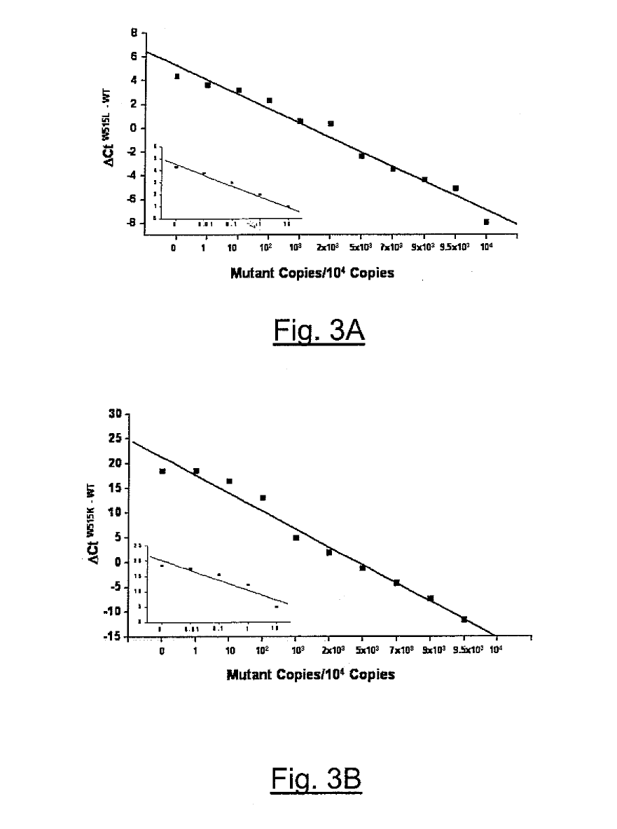

The amount of amplified DNA is expressed as Ct or threshold cycle (FIG. 3-1). It is the first cycle in which a statistically relevant change of the Rn is detected. The threshold is defined as the average standard deviation of Rn for early cycles multiplied by a correction factor. The threshold cycle is obtained when the Measuring system (Sequence Detection System) starts detecting the increase of the signal associated with an exponential growth of the PCR product, as shown in the Figure. The threshold cycle proportionally reduces as the number of sequence copies detected by the probe present in the sample grows.

Primers

Primers are two DNA oligonucleotides obtained through synthesis in solid phase, typically of 18-30 base pairs (bp), whose sequence is perfectly compatible with two DNA segments flanking the regions of the DNA to be amplified. One of the two primers is complementary to the sequence of the DNA strand containing the sequence in question (forward primer), the other is complementary to the antiparallel sequence of the DNA (reverse primer).

Thus, the primers are essential components of the PCR reaction in that they are responsible for the specificity of the amplified fragment; their sequence is selected according to the degree of complementarity with the target sequence, in such a manner to reduce to the minimum the possibility that they may bind in a non-specific manner, and even only partially, to other partially similar genetic sequences but which are not relevant, and thus should not be amplified.

The identification of the optimal sequence of the primers is performed knowing the sequence of the gene in question, usually available from public data bases as described above.

Once the researcher has identified the gene segment to be amplified, he proceeds to select the primers, also this through programmes freely available in public access sites, such as ENTREZ NUCLEOTIDE, or others such as PRIMER EXPRESS (provided by Applied Biosystems).

Important, but not exhaustive, aspects to be taken into account when designing and choosing primers are: the appropriate length of the amplicon, which allows identification thereof regarding the chosen detection technique; a similar content of Cytosine (C) and guanine (G) bases in the two primers in such a manner that the dissociation temperature of the primers (melting temperature) is similar; the length of the primers and the fact that their sequence does not generate non-specific pairings relative to each other upon mixing, instead of the DNA target sequence (so-called formation of dimers).

The primers used in the present invention for amplifying—through RTQ-PCR—the MPL gene fragment containing the codon corresponding to amino acid 515 (codon 515) have sequence 5′-3′ SEQ ID NO:1 (forward) and SEQ ID NO: 2 (reverse).

The primers used for direct sequencing and for cloning the MPL gene fragment of interest have 5′-3′ SEQ ID NO: 6 (forward sequence); SEQ ID NO: 7 (reverse); SEQ ID NO: 8 (forward) and SEQ ID NO: 9 (reverse)

Probes and LNA Technology

A fundamental aspect of this invention is represented by the design and by the optimisation of probes which are specific for the two mutations of codon 515 of the MPL gene. As a matter of fact, the present inventors discovered that use of traditional DNA probes, produced by means of traditional chemistry, did not allow obtaining substantial results due to the low hybridization specificity at ideal temperature, calculated according to the respective melting temperature, and verified in an experimental manner.

Therefore, the probes used in the present invention were produced by means of a chemical modification according to the LNA technology, to make them ideal for the preset purpose.

LNA®s (Locked Nucleic Acid) are innovative oligonucleotides with higher hybridization characteristics and greater biostability with respect to conventional oligonucleotides used as probes or primers in the PCR reactions, both conventional and Real Time. The LNA nucleotide is an analogous nucleotide containing an oxygen bridge bond in 2′ with carbon in 4′, as shown in formula I below. This bridge limits the flexibility of the ribofuranose ring and creates a rigid structure with an endo-configuration locked on C3, which confers greater hybridation capacity and greater stability of the molecule (Latorra D, Campbell K, Wolter A, Hurley J M. Enhanced allele-specific PCR discrimination in SNP genotyping using 3′ locked nucleic acid (LNA) primers. Hum Mutat. 2003; 22:79-85).

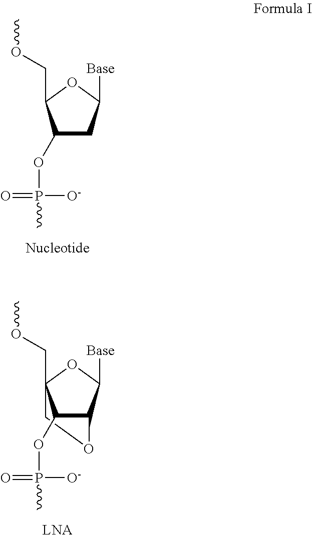

As observable from the comparison between the formulae of a natural nucleotide and an LNA modified nucleotide, the latter is characterised by the presence of a bridge bond between oxygen in 2′ and carbon in 4′, which is responsible for the functional properties of the LNA nucleotide in terms of greater stability, due to the structural rigidity, and the increase of the melting temperature (Tm) value of the nucleotide sequence in which it is inserted.

The synthesis cycle of the LNA nucleotides (which can be obtained on sale) consists in a series of reactions similar to the synthesis of DNA monomers with difference being that the polymerisation step of LNA monomers is much slower with respect to that of non-modified DNA (8 minutes against 90 seconds). Application of this technology in the probe synthesis to detect the specific DNA sequences subjected to amplification by means of PCR technique has numerous advantages: increasing the thermal stability and hybridization specificity, greater accuracy in gene quantification and allele discrimination (meaning, capacity to discriminate between a normal sequence (normal or wild-type allele) and a sequence mutated in one or more nucleotide bases (mutated allele)), and greater flexibility in defining the most suitable sequence. The thermal stability of the duplex is extensively documented by chemical/physical studies which show an increase of the melting temperature (Tm) up to 8° C. with respect to the value obtained using the classic DNA probes.

Furthermore, it was observed that introducing the LNA probe into a quantitative PCR reaction increases the stability of the duplex (that is of the double DNA strand) which is formed between the original DNA sequence and the sequence that is synthesised during the PCR cycles, and it improves the specificity for the target sequence. Further advantages lie in that non-specific fluorescence is lowered and the ratio between specific fluorescence signal and the background noise increases with respect to using probes having the same identical sequence but produced by means of conventional chemical reaction.

Due to these characteristics, the LNA probes were particularly useful in techniques requiring high specificity, such as SNP (single nucleotide polymorphism) analysis, and hence particularly useful for mutational analysis according to the present invention. As a matter of fact, object of such analysis is that of allowing to discriminate between the normal sequence of the cordon 515 of the MPL gene (TGG, coding for the tryptophan amino acid) and the mutated sequences respectively of type L (TTG, coding for the leucine amino acid) or K (AAG, coding for the lysine amino acid) that is the substitution of only 1 base in case of mutation of type W>L and two bases in case of mutation of type W>K.

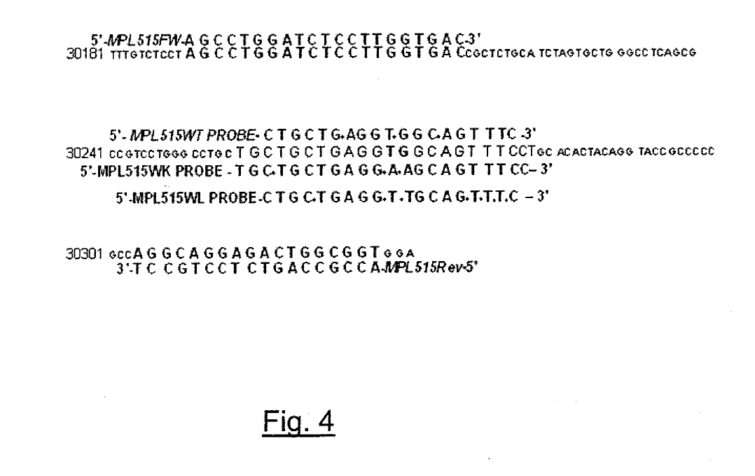

The probes used in the invention are indicated in FIG. 4 and are comprised in the zone between nucleotide 30241 and 30300 of the genome sequence of the MPL gene. Such probes comprise at least one nucleotide, but preferably two, three, four, five or more LNA nucleotides. In particular, the specific probe for the non-mutated gene (Wild-Type) (MGB Probe), has the sequence (SEQ ID NO: 3) CTGCTGAGGTGGCAGTTTC and contains at least one or more LNA modified nucleotides. Preferably, the LNA probe contains three modified nucleotides which, in the most preferred embodiment, are indicated on SEQ ID NO3 in bold type preceded by an asterisk: CTGCTG*AGGT*GGC*AGTTTC.

The specific probe for the mutated allele 515W>L (MGB probe) has sequence (SEQ ID NO: 4) CTGCTGAGGTTGCAGTTTC and contains at least one or more LNA modified nucleotides. Preferably, the LNA probe contains five modified nucleotides which, in the most preferred embodiment, are indicated on SEQ ID NO 4 in bold type preceded by an asterisk: CTGC*TGAGG*T*TGCAG*T*TTC.

Lastly, the specific probe for the mutated allele 515W>K (MGB probe) has sequence (SEQ ID NO: 5) TGCTGCTGAGGAAGCAGTTTCC and contains at least one or more LNA modified nucleotides. Preferably, the LNA probe contains three modified nucleotides which, in the most preferred embodiment, are indicated SEQ ID NO 5 in bold type preceded by an asterisk: TGC*TGCTGAGG*A*AGCAGTTTCC.

Such probes are labelled for example with 5,6 carboxyfluorescein at 5′ end and with the Black Hole Quencher-1 (BHQ-1) at 3′ end.

Procedure

In order to calibrate the system and obtain quantitative evaluations capable of allowing to express the possible presence of the mutated allele as a percentage of the wt one, a plasmid DNA solution, made up of an MPL wild-type plasmid and mutated MPL 515W>L or 515W>K, are used in the amplification reactions as an internal reference in amounts in the range of 100%-0% of mutated allele. By evaluating their ΔCₜ standard curves can be obtained from which the amount of mutated allele present can be derived if the sample carries the mutated gene.

Preparation of the standard curve which allows calculating the percentage of mutated allele is performed as described hereinafter. A fragment of the MPL gene containing the region where the mutations of codon 515 are located is obtained through plasmid cloning by means of conventional procedures after having identified a suitable pair of Forward and Reverse primers (SEQ ID NO: 8 & No: 9). The suitability of the cloned fragment of the MPL gene, when constructing the reference curve, was evaluated through bidirectional direct sequencing reaction after amplification by means of PCR, and using a specific pair of Forward and Reverse primers (SEQ ID NO: 6 & No: 7). Serial dilutions of mutated plasmid in a ratio in the range of 0.01%-100% in mixture of 99.99% to 0% plasmid of allele wild-type were the prepared. The calibration curves of the mutated alleles W>L and W>K are indicated in FIGS. 6 A and B.

RTQ PCR Procedure

In order to evaluate the MPL mutation three different PCR reactions are prepared for each sample. The same two common primers flanking the regions of the MPL gene of interest (forward primer and reverse primer, (SEQ ID NO: 1 & No: 2) are used in all of them. On the contrary, the fluorogenic probe is different for each of the three reactions: one complementary to wild type sequence (SEQ ID NO: 3), one specific for the MPLW515L (SEQ ID NO: 4) mutation and one for the MPLW515K (SEQ ID NO: 1 & No: 2) mutation. 40 ng of DNA are used for each sample. The reaction mix contains the buffer with the optimal concentration of magnesium chloride, deoxynucleotide triphosphates, and polymerase DNA. The following PCR protocol is performed: denaturation at 50° C. for 2 minutes and 95° C. for 10 minutes and then amplification (95° C. for 15 seconds, 58° C. for 1 minute in case of the MPLW515T and MPLW515L per, 62° C. for MPLW515K per) for 40 cycles. Both the StepOne Real Time PCR System machine and the ABI Prism 7300 (Applied Biosystem) machine were used with entirely identical results. For each sample, the ΔCₜ—which is given by the difference between the average threshold cycle of the reaction which uses the probe for the mutated allele and the average threshold cycle of the reaction with the probe for the wt allele (ΔCₜ=mutated Cₜ−wt Cₜ) is calculated. For example, a mutated sample, in a reaction using a specific probe for the present mutation, shall definitely have a much lower threshold cycle with respect to a sample that does not have such mutation, which—on the contrary—shall have a lower value of the threshold cycle in the test tube containing the wt probe. Therefore, the ΔCₜ of a mutated sample shall be proportionally reduced, with respect to a normal wild-type sample, as the mutated allele represented in percentage with respect to the normal one increases.

The samples that resulted mutated with this technique were subjected to sequencing with ABI PRISM 3730DNA Analyzer (Applied Biosystems, Foster City, Calif., USA), to confirm the presence of the alteration in the sequence.

Kit

Performance of the methods of the invention implies the following reagents and aspects: a set of molecular probes identified and optimised for quantitative detection of the 515 W>L and 515W>K mutations of the MPL gene in a Real-Time quantitative PCR (RTQ-PCR) system, and which are made up of:
a pair of Forward and Reverse primers, which flank the region of the MPL gene where the mutations (SEQ ID NO: 1 & No: 2) are located;
a specific LNA probe for the wild-type sequence of the MPL gene (SEQ ID NO: 3)
a specific LNA probe for the mutated 515 W>L sequence (SEQ ID NO: 4)
a specific LNA probe for the mutated 515 W>K sequence (SEQ ID NO: 5).

Such reagents are provided in form of kits of components containing at least the modified probes, but preferably also the primers and all the reagents required to implement the analytical method, such as: solution buffered with a suitable concentration of salts, in particular magnesium chloride; cytosine, guanine, adenine and thymine deoxyribonucleotide triphosphates, and enzyme, the thermostable DNA polymerase.

Applications

The present invention finds application in the molecular diagnostics of chronic myeloproliferative syndromes. As a matter of fact, the presence of mutations of the MPL gene was considered to be a major diagnostic criterion for the diagnosis of chronic myeloproliferative syndrome, such as Essential Thrombocythemia or Primary Myelofibrosis, in the new diagnostic criteria proposed by the World Health Organization.

Advantages offered by the invention with respect to the current state of the art are linked to the fact that the new method designated in the invention allows identifying the presence of the mutation in samples of Genomic DNA in a much more sensitive manner with respect to the conventional method based on direct sequencing or melting curves; it is less expensive, and it is based on a RTQ-PCR technology accessible to most of the diagnostic laboratories. Therefore, the invention may allow a more capillary application of the research of mutations of the MPL gene in patients affected by chronic myeloproliferative diseases. In particular, its sensitivity at least 10-50 times greater with respect to direct sequencing, and 3-10 times greater with respect to the melting curves, shall allow identifying as mutated MPL even patients with low allele load, lower than 15-10%, otherwise not identifiable using the direct sequencing method.

Furthermore, according to data already published by our group relevant to patients affected by Primary Myelofibrosis—using a method for direct sequencing of the gene—and data still to be completed in patients with Essential Thrombocythemia using this invention developed by our group, there is the possibility that the amount of mutated allele also acquires a prognostic value. From this point of view, it is particularly important that there be a method capable of providing a quantitative estimation of the amount of mutated allele; this information was merely semi-quantitative and scarcely reproducible, using the standard direct gene sequencing technique.

Lastly, the high sensitivity of the procedure described makes it particularly suitable for studying the effects of innovative drugs, currently being evaluated in international experiments, in that it could allow accurate measurement of the so-called “minimal residual disease”, i.e. the percentage—even very low—of mutated MPL alleles left in the patient after the therapy (for analogy with other pathologies, such as chronic myelogenous leukemia, in which the RTQ-PCR systems are also used to monitor the patient's response to the therapy).

### Experimental Examples

### Reagents, Material and Dedicated Instrument

Ficoll Hypaque (ex, Lymphoprep)
Lysing solution (8 mM ammonium chloride, 10 mM potassium bicarbonate, 1 mM edta tetrasodium)
Saline buffer (D-PBS, Dulbecco's phosphate buffered saline without calcium and magnesium, Euroclone, Europe)
Genomic DNA Extraction system (QIAmp DNA Blood kit, Quiagen, Germany)
Taqman Universal MasterMix
Primers (SEQ ID NO: 1 & No: 2)
LNA DFLP probes: (SEQ ID NO: 3, No:4 & No: 5)
RNAse free water
DNAse/RNAse free tips with filter
Plates for RealTime PCR
Nanodrop (NanoDrop Technologies, USA) spectrophotometer
Apparatus for RT-PCR, StepOne Real Time PCR machine, Applied Biosystems
Apparatus for RT-PCR ABI-PRISM 7300, Applied Biosystems

Biological Material to be Tested:

Genomic DNA extracted from peripheral blood granulocytes

Step 1. Separating Granulocytes

Peripheral blood samples, collected in EDTA or sodium citrate, are stratified on Lymphoprep according to a ratio of about 10-15 ml of blood on 10 ml of lymphoprep and subsequently centifugated at 1600 rpm for 30 min at room temperature. After having suctioned the supernatant and eliminated the mononucleated fraction, the lower part of the gradient is resuspended in about 40 ml of ammonium chloride to eliminate possible red cells contaminations, centrifugation is performed again at 1500 rpm for 8-10 min at 8-10° C., then the supernatant is suctioned. Washing is performed in a phosphate buffer (D-PBS, Dulbecco's phosphate buffered saline without calcium and magnesium, Euroclone, Europe) The granulocytes pellet, after another resuspension in lysing solution, is subjected to one/two washings in saline-phosphate buffer pH 7.4 (PBS) and it is directly frozen at −20° C. for the subsequent purification of the DNA.

Step 2. DNA Extraction

Genomic DNA is extracted from the granulocytes according to protocol QIAmp DNA Blood kit (Quiagen, Germany). In brief, the granulocyte pellet is resuspended in 200 μl of PBS and 20 μl of Proteinase K solution and 200 μl of Buffer AL are added. After incubation at 56° C. for 10 minutes 200 μl of ethanol are added and the mix is transferred to a QIagen column and centrifugated at 6000 rpm for 2 minutes and subsequently at 12000 rpm for 1 minute. After transferring the column onto a new test tube 500 μl of BUFFER AW1 are added. After centrifugation at 8000 rpm for 1 minute, 500 μl of BUFFER AW2 are added. After centrifugation at 12.000 rpm for 4 minutes, 100 μl of BUFFER AE are added and the mix is left for 5 minutes at room temperature. Lastly, after centrifugation at 12.000 for 1 minute the eluate obtained is transferred into a sterile test tube. Dosage and determination of the purity of the extracted DNA are performed through spectrophotometer reading using the Nanodrop system.

Step 3. Setting Up the Real-Time PCR Reaction

The reaction provides for the amplification of Genomic DNA, extracted from granulocytes, in three different reactions, in each one of which one and only specific probe (SEQ ID NO: 3-4 & No: 5) is used.

Primers and Probes Dilutions: primers and probes are resuspended in a stock solution of 100 μmol, and they are used in the amplification reaction at the final concentration of 300 nmol and 200 nmol, respectively for the primers and probes.

20 ng in duplicate for a total of 40 ng of DNA were used for each sample. Furthermore, the reaction mix contained Taqman 2× Universal Pcr Master Mix containing the polymerase DNA, dNTP and the Rox probe. Then the following PCR protocol was followed: denaturation at 50° C. for 2 minutes and 95° C. for 10 minutes and then amplification (95° C. for 15 seconds, 58° C. for 1 minute in the case of the MPLW515T and MPLW515L per, 62° C. for MPLW515K per) for 40 cycles using, with entirely identical results, the ABI PRISM7300 or StepOne Real Time PCR System (Applied Biosystems) apparatus.

Step 4. Analysis of the Reaction Results.

The ΔCₜ which is given by the difference between the average threshold cycle of the reaction which uses the probe for the W>L or W>K mutated alleles and the average threshold cycle of the reaction with the specific probe for the non-mutated allele (wild-type) (ΔCₜ=Cₜ mutated−Cₜ wt) was calculated for each sample.

Other Procedures

DIRECT SEQUENCING. About 100 ng of granulocyte DNA were amplified for 40 cycles (30 seconds at 95° C., 30 seconds at 62° C., and 30 seconds at 72° C.; final extension at 72° C. for 7 minutes) in a total reaction volume of 40 μl containing: 1 U of Taq Gold DNA Polymerase, 1 μmol/l of the forward (SEQ ID NO: 6) and reverse (SEQ ID NO: 7) primers, 200 mM of each dNTP, 2 mM of MgCl2, and 5 μl of reaction buffer 10× (10 mM Tris-HCl pH 8.3, 50 mM KCl). The reaction product, an amplicon of 248 bp, is then analysed through electrophoresis on agarose gel at 2%, purified, and subjected to a bidirectional sequencing.

Cloning the Fragment of the MPL Gene Containing Codon 515 in Plasmid Vector

The cloning procedure provides for an amplification reaction by means of PCR using the SEQ ID NO: 8 and NO:9 specific primers, which produce an amplicon 277 bp. Three different amplicons corresponding to the wild-type allele, mutated 515W>L and 515W>K were prepared, the latter obtained from mutated patients previously identified through direct bidirectional sequencing. The sequence of each amplicon was confirmed through bidirectional direct sequencing. The cloning reaction was performed by means of the PCR II TOPO vector (InVitrogen) system, according to the instructions of the manufacturing firm. A mix containing the PCR, TOPO Vector product and a saline solution facilitating the introduction of the fragment into the plasmid was prepared. The mix was left to incubate for 5 min at room temperature. Subsequently, the transformation step of chemical type in One Shot E. coli occurred. 2 μl of TOPO Cloning mix were added to a test tube of bacteria which were thawed in ice and then stirred gently. An incubation in ice was performed for 15 minutes and the test tube was rapidly transferred for 30 seconds at 42° C. without stirring. Then, a suitable amount of bacteria was added to 250 μl of SOC medium previously kept at room temperature. The test tube containing the mixture was maintained at 37° C. in the incubator for 1 hour under stirring. 50 μl of bacteria were plated on a selective plate with agar containing ampicillin preheated at 37° C. Incubation was then performed at 37° C. overnight. The following day, only the white colonies which had been formed and which carry the plasmid containing the insert were collected, leaving out the blue colonies. Each single colony was grown in an LB medium at 37° C. under constant stirring. The bacterial cells were thus lysed and the plasmid DNA is purified using the PureLink Quick Plasmid Miniprep Kit (Invitrogen, Groningen, Netherlands). The plasmid DNA was eluated in ET, quantified spectrophotometrically and stored at −20° C. The sequence was determined through bidirectional direct sequencing. The number of plasmid copies per pg was evaluated taking into account the weight of the vector and insert.

Results

60 normal subjects (blood donors) and more than 1000 patients affected by Primary Myelofibrosis or Essential Thrombocythemia were evaluated using the method according to the invention. No normal subject was considered a mutation carrier in any of the cases, hence confirming the specificity of the method. Among the more than 40 patients with MPD identified as mutated by means of this technology, the presence of the mutation was confirmed through direct sequencing in 37/40 cases, while in the remaining three, in which the presence of the mutation was confirmed after plasmid cloning and analysis of single plasmid clones, the negativity resulting from direct sequencing was attributed to the low percentage of mutated allele present (in two cases of the W>L type, of W>K type), about 1-3%, hence below the discriminating capacity of the direct sequencing. This observation confirms the greater diagnostic sensitivity of the invention, which hence shall identify further cases of patients having the MPL mutations with respect to the results of conventional sequencing.

Lastly, we calculated, in more than 3000 RTQ-PCR reactions performed, that the inter-assay intra-assay values are very low. In the case of the intra-assay variability, this was lower than 2% for the wt allele, 1% for the W>L allele and <1% for the W>K allele. In the case of the inter-assay value, this amounted to 2.5% for the wt allele, 2% for the W>L allele, 0.8% for the W>K allele. This was considered an evidence of the repeatability and reliability of the procedure.

SEQUENCE LISTING

| Primer | Sequence 5′ ----> 3′ |
|---|---|
| SEQ ID NO: 1 | AGCCTGGATCTCCTTGGTGAC |
| Forward Primer for Real-Time PCR | |
| SEQ ID NO: 2 | ACCGCCAGTCTCCTGCCT |
| Reverse Primer for Real-Time PCR | |
| SEQ ID NO: 3 | CTGCTGAGGTGGCAGTTTC |
| LNA-modified MGB Probe, | |
| fluorochrome labelled, specific for | |
| not-mutated sequence (Wild-Type) | |
| SEQ ID NO: 4 | CTGCTGAGGTTGCAGTTTC |
| LNA-modified MGB Probe, | |
| fluorochrome labelled, specific for | |
| the mutated sequence 515W > L | |
| SEQ ID NO: 5 | TGCTGCTGAGGAAGCAGTTTCC |
| LNA-modified MGB Probe, | |
| fluorochrome labelled, specific for | |
| the mutated sequence 515W > K | |
| SEQ ID NO: 6 | TAGCCTGGATCTCCTTGGTG |
| Forward Primer for direct | |
| sequencing reaction | |
| SEQ ID NO: 7 | AGAGGTGACGTGCAGGAAGT |
| Reverse Primer for direct | |
| sequencing reaction | |
| SEQ ID NO: 8 | TGGGCCGAAGTCTGACCCTTT |
| Forward Primer for cloning the MPL | |
| gene fragment of interest | |
| SEQ ID NO: 9 | AGAGGTGACGTGCAGGAAGTGGCGAAGC |
| Reverse Primer for cloning the MPL | |
| gene fragment of interest | |

1-14. (canceled)

15. A labelled genetic probe configured for detecting a mutation on codon 515 of MPL gene and quantitatively determining a corresponding mutated allele of said MPL gene, wherein the labelled genetic probe contains at least one LNA nucleotide.

16. The labelled genetic probe of claim 15, wherein codon 515 of the MPL gene is TGG and codon 515 of the mutated allele is TTG or AAG.

17. The labelled genetic probe of claim 15, wherein the labelled genetic probe has nucleotide sequence SEQ ID: 3, SEQ ID 4 or SEQ ID 5.

18. The labelled genetic probe of claim 17, wherein the labelled genetic probe has nucleotide sequence SEQ ID NO: 3 and the at least one LNA nucleotide comprises nucleotides in position 7, 11 and 14.

19. The labelled genetic probe of claim 17, wherein the labelled genetic probe has nucleotide sequence SEQ ID NO: 4 and the at least one LNA nucleotide comprises nucleotides in position 5, 10, 11, 16 and 17.

20. The labelled genetic probe of claim 17, wherein the labelled genetic probe has nucleotide sequence SEQ ID NO: 5 and the at least one LNA nucleotide comprises nucleotides in position 4, 12 and 13.

21. A primer for amplifying a fragment of MPL gene, the fragment comprising codon 515 of the MPL gene, the primer having nucleotide sequence SEQ ID: 1, SEQ ID 2, SEQ ID 6, SEQ ID 7 or SEQ ID 9.

22. A method for in vitro mutational analysis directed to detect mutations on codon 515 of MPL gene and to quantitatively determine a corresponding mutated allele of said MPL gene, the method comprising
amplifying by RTQ-PCR a genomic DNA containing the MPL gene in presence of one or more labelled genetic probes of claim 15.

23. The method of claim 22, wherein the one or more labelled genetic probes has nucleotide sequence SEQ ID: 3, SEQ ID 4 or SEQ ID 5.

24. The method of claim 23, wherein the labelled genetic probe having SEQ ID NO: 3 comprises an LNA nucleotide in position 7, 11 and 14; the labelled genetic probe having SEQ ID NO: 4 comprises an LNA nucleotide in position 5, 10, 11, 16 and 17; and the labelled genetic probe having SEQ ID NO: 5 comprises an LNA nucleotide in position 4, 12 and 13.

25. The method of claim 22, wherein the amplifying is performed using as a forward primer an oligonucleotide having sequence SEQ ID 1 and as a reverse primer an oligonucleotide having sequence SEQ ID 2.

26. The method of claim 22, wherein the amplifying is performed using primers and probes at concentration of 300 nmol and 200 nmol respectively, master mix (AB) in dilution 2×.

27. The method of claim 22, wherein the amplifying results in an amplified MPL gene fragment suitable for molecular diagnosis or for molecular prognosis of chronic myeloproliferative diseases.

28. Kit of parts for in vitro mutational analysis of MPL gene, the kit comprising:
a specific probe for wild-type sequence of the MPL gene containing at least one LNA nucleotide;
a specific probe for mutated sequence 515 W>L containing at least one LNA nucleotide; and
a specific probe for mutated sequence 515 W>K containing at least one LNA nucleotide;
for use in the method of claim 22.

29. The kit according to claim 28, further comprising a pair of forward and reverse primers flanking a region of the MPL gene where codon 515 is located.

30. The kit according to claim 29, comprising probes having nucleotide sequence SEQ ID: 3, SEQ ID 4 and SEQ ID 5; and primers having sequence SEQ ID 1 and SEQ ID 2.

31. The kit according to claim 30, wherein the probe having SEQ ID NO: 3 comprises an LNA nucleotide in position 7, 11 and 14, the probe having SEQ ID NO: 4 comprises an LNA nucleotide in position 5, 10, 11, 16 and 17, and the probe having SEQ ID NO: 5 comprises an LNA nucleotide in position 4, 12 and 13.

32. An MPL gene fragment amplified using primers of sequence SEQ ID 8 and SEQ ID 9.

33. A cloning plasmid containing the MPL gene fragment of claim 32.
